(19) United States
(12) Patent Application Publication
(10) Pub. No.: US 2026/0345367 A1
(43) Pub. Date: Nov. 5, 2026

(54) FOAMED PAD

(71) Applicant: BAUERFEIND AG, Zeulenroda-Triebes (DE)

(72) Inventors: Robin WÖLLNER, Zeulenroda-Triebes (DE); Dominique PANZER, Zeulenroda-Triebes (DE); Beatrix BAUERFEIND-JOHNSON, Zeulenroda-Triebes (DE)

(21) Appl. No.: 19/488,054

(22) PCT Filed: May 15, 2024
(86) PCT No.: PCT/EP2024/063453

§ 371 Date: Nov. 25, 2025

(30) Foreign Application Priority Data
May 31, 2023 (DE) 10 2023 205 103.6

(51) Int. Cl. A61F 5/30

(52) U.S. Cl. CPC A61F 5/30

(57) ABSTRACT

The present invention relates to pad comprising a foamed pad main body made of thermoplastic elastomer (TPE) with a density of at least 0.4 g/cm³ and at most 0.8 g/cm³. The present invention also relates to orthopedic aids or sports aid devices comprising a pad according to the invention.

The present invention relates to pad comprising a foamed pad main body made of thermoplastic elastomer (TPE) with a density of at least 0.4 g/cm³ and at most 0.8 g/cm³. The present invention also relates to orthopedic aids or sports aid devices comprising a pad according to the invention.

Pads are devices that are generally used for medical or sports support purposes for local compression or for compressing or holding tissue in place. They can also be used for padding. Orthopedic aids or sports aid devices are often equipped or upgraded with such pads. Pads are made of elastic materials, often silicone or polyurethane. The pads can be embedded in a covering. Pads are known in various embodiments and for various prophylactic and therapeutic applications. Various pads are disclosed, for example, in DE 27 22 563C2, EP 0 598 291A1, and EP 0 600 218 A2.

Pads must have a specific Shore hardness in order to function effectively as pressure bodies. Furthermore, pads should comprise specific viscoelastic behavior in order to adapt to the user's body as effectively as possible and to fulfill their intended purpose optimally.

Due to the material used, silicone or TPE pads with the desired Shore hardness are comparatively heavy in the prior art. The manufacturing costs are also comparatively high.

The technical problem underlying the invention is the provision of a pad that comprises a suitable Shore hardness and viscoelastic properties, but is lighter than comparable pads in the prior art and/or cheaper or even cheaper to manufacture.

The present invention solves the underlying technical problem by means of a pad according to claim 1.

The present invention relates to a pad comprising a foamed pad main body, characterized in that the pad main body is formed from thermoplastic elastomer (TPE) as the main component, comprises a hardness of at least 20 Shore OO and at most 45 Shore OO, and comprises a density of at least 0.4 g/cm³ and at most 0.8g/cm³.

Preferred is a pad comprising a foamed pad main body, characterized in that the pad main body is formed from thermoplastic elastomer (TPE) as the main component, comprises a hardness of at least 20 Shore OO and at most 45 Shore OO, and comprises a density of at least 0.4 g/cm³ and at most 0.8g/cm³, comprises a storage modulus of at least 0.15 MPa and at most 0.5 MPa in a temperature range of at least 18 °C and at most 40 °C, comprises a loss modulus of at least 0.015 MPa and at most 0.03 MPa in a temperature range of at least 10 °C and at most 40 °C, and comprises a loss factor tan delta of at least 0.05 and at most 0.25 in a temperature range of at least 10 °C and at most 40 °C.

Such pads advantageously comprise a suitable Shore hardness and a comparatively low density. It has thus been shown that pads made of thermoplastic elastomer (TPE) as the main component, with this low density of at least 0.4 g/cm³ and at most 0.8 g/cm³, are soft yet dimensionally stable enough to achieve the desired effect when wearing the pad. Uniform pore formation can also be achieved during foaming during manufacture. By modulating the values of storage modulus, loss modulus, loss factor, density, Shore hardness, and bubble size, a foam can be provided that meets the cushioning and support requirements of orthopedic technology and sports technology for an optimized effect on the user.

The thermoplastic elastomer forms the main component of the pad main body. This means that the thermoplastic elastomer is the key body-forming material of the pad main body, but that other materials and substances, such as oils and waxes, may also be comprised in the pad main body, even in large quantities. For example, thermoplastic elastomers such as styrene-ethylene-butylene-styrene are processed as a gel consisting of a mixture that, in addition to styrene-ethylene-butylene-styrene, comprises up to more than 90 wt % of oil, for example a white oil mixture. Even when this mixture is processed into a foamed article, in this case the pad main body, the main component is the thermoplastic elastomer.

Preferably, the pad main body according to the invention comprises an oil or an oil mixture. Preferably, the pad main body according to the invention comprises an oil or an oil mixture in a quantity of 0 wt % to 95 wt %. Preferably, the pad main body according to the invention comprises an oil or an oil mixture in a quantity of 1 wt % to 95 wt %. Preferably, the pad main body according to the invention comprises an oil or an oil mixture in a quantity of 50 wt % to 95 wt %. Preferably, the pad main body according to the invention comprises an oil or an oil mixture in a quantity of 51 wt % to 95 wt %. Preferably, the oil or oil mixture is a white oil.

Preferably, the thermoplastic elastomer is selected from the group consisting of styrene block copolymers (TPS), thermoplastic polyurethanes (TPU), thermoplastic vulcanizates (TPV), thermoplastic polyamide elastomers (TPA), thermoplastic copolyester elastomers (TPC), and mixtures thereof.

Preferred is a pad comprising a foamed pad main body, characterized in that the pad main body is formed from styrene block copolymer (TPS) as the main component, comprises a hardness of at least 20 Shore OO and at most 45 Shore OO, and comprises a density of at least 0.4 g/cm³ and at most 0.8g/cm³.

Preferred is a pad comprising a foamed pad main body, characterized in that the pad main body is formed from styrene block copolymer (TPS) as the main component, comprises a hardness of at least 20 Shore OO and at most 45 Shore OO, and comprises a density of at least 0.4 g/cm³ and at most 0.8g/cm³, a storage modulus of at least 0.15 MPa and at most 0.5 MPa in a temperature range of at least 18 °C and at most 40 °C, comprises a loss modulus of at least 0.015 MPa and at most 0.03 MPa in a temperature range of at least 10 °C and at most 40 °C, and comprises a loss factor tan delta of at least 0.05 and at most 0.25 in a temperature range of at least 10 °C and at most 40 °C.

In a preferred embodiment, the thermoplastic elastomer is mainly a styrene block copolymer (TPS). Surprisingly, it was found that styrene block copolymers are especially well suited for manufacturing pads with the desired Shore hardness and low density. When using styrene block copolymers, for example styrene/butadiene-styrene (SBS) and/or styrene-ethylene-butylene-styrene (SEBS), very uniform pore formation can be achieved during foaming during manufacturing.

Preferably, the pad consists mainly of a styrene block copolymer (TPS).

In a preferred embodiment, the thermoplastic elastomer is a styrene block copolymer (TPS; also known as TPE-S).

Preferably, the pad consists mainly of at least one styrene block copolymer (TPS) and an oil or an oil mixture.

Preferably, the pad main body according to the invention, made of a styrene block copolymer (TPS), comprises an oil or an oil mixture. Preferably, the pad main body according to the invention, made of a styrene block copolymer (TPS), comprises an oil or an oil mixture in a quantity of 0 wt % to 95 wt %. Preferably, the pad main body according to the invention, made of a styrene block copolymer (TPS), comprises an oil or an oil mixture in a quantity of 1 wt % to 95 wt %. Preferably, the pad main body according to the invention, made of a styrene block copolymer (TPS), comprises an oil or an oil mixture in a quantity of 50 wt % to 95 wt %. Preferably, the pad main body according to the invention, made of a styrene block copolymer (TPS), comprises an oil or an oil mixture in a quantity of 51 wt % to 95 wt %. Preferably, the oil or oil mixture is a white oil.

In a preferred embodiment, the styrene block copolymer (TPS) is on the basis of a copolymer selected from the group consisting of styrene/butadiene-styrene (SBS), styrene-ethylene-butylene-styrene (SEBS), styrene-ethylene/propylene-styrene (SEPS), styrene-ethylene-ethylene-propylene-styrene block copolymer (SEEPS), styrene-isoprene-styrene (SIS), methyl methacrylate-acrylonitrile-butadiene-styrene (MABS), methyl methacrylate-butadiene-styrene (MBS) and mixtures thereof. Other suitable styrene block copolymers are known to a person skilled in the art.

In a preferred embodiment, the styrene block copolymer (TPS) is formed on the basis of styrene/butadiene styrene (SBS) and/or styrene-ethylene-butylene styrene (SEBS).

In a preferred embodiment, the styrene block copolymer (TPS) is formed on the basis of styrene/butadiene styrene (SBS) or styrene-ethylene-butylene styrene (SEBS).

In a preferred embodiment, the styrene block copolymer (TPS) is formed on the basis of styrene/butadiene styrene (SBS).

In a preferred embodiment, the styrene block copolymer (TPS) is formed on the basis of styrene-ethylene-butylene-styrene (SEBS).

In a preferred embodiment, the styrene block copolymer (TPS) is styrene/butadiene-styrene (SBS) and/or styrene-ethylene-butylene-styrene (SEBS).

In a preferred embodiment, the styrene block copolymer (TPS) is styrene/butadiene-styrene (SBS). In a preferred embodiment, the styrene block copolymer (TPS) is styrene-ethylene-butylene-styrene (SEBS). In a preferred embodiment, the styrene block copolymer (TPS) is styrene/butadiene-styrene (SBS) and styrene-ethylene-butylene-styrene (SEBS).

Preferred is a pad comprising a foamed pad main body, characterized in that the pad main body is formed from styrene/butadiene-styrene (SBS) or styrene-ethylene-butylene-styrene (SEBS) as the main component, comprises a hardness of at least 20 Shore OO and at most 45 Shore OO, and comprises a density of at least 0.4 g/cm³ and at most 0.8g/cm³.

Preferred is a pad comprising a foamed pad main body, characterized in that the pad main body is formed from styrene/butadiene-styrene (SBS) or styrene-ethylene-butylene-styrene (SEBS) as the main component, comprises a hardness of at least 20 Shore OO and at most 45 Shore OO, and comprises a density of at least 0.4 g/cm³ and at most 0.8 g/cm³, comprises a storage modulus of at least 0.15 MPa and at most 0.5 MPa in a temperature range of at least 18 °C and at most 40 °C, comprises a loss modulus of at least 0.015 MPa and at most 0.03 MPa in a temperature range of at least 10 °C and at most 40 °C, and comprises a loss factor tan delta of at least 0.05 and at most 0.25 in a temperature range of at least 10 °C and at most 40 °C.

Suitable methods for manufacturing foamed bodies, such as pad main bodies, from thermoplastic elastomers (TPE), for example styrene block copolymers (TPS), are known to the person skilled in the art. A typical method is described in principle in the example given in this application.

It is also generally known to foam thermoplastic elastomers (TPE) using propellants. WO2004108811A1, for example, describes methods for manufacturing expandable thermoplastic elastomers.

Preferably, the pad main body is foamed during manufacture. Preferably, the pad main body is formed from foamed thermoplastic elastomer (TPE) as the main component. Preferably, the pad main body is formed from foamed styrene block copolymer (TPS) as the main component.

Surprisingly, it was found that by foaming thermoplastic elastomers, pad main bodies and pads with the desired Shore hardness and low density can be manufactured especially well and inexpensively. Since foaming thermoplastic elastomers, particularly with a propellant, is known to the person skilled in the art as a common and inexpensive process, it can also be used well in the present case, wherein, surprisingly, this common process can be used to manufacture pads that meet the requirements for a pad and, in particular, comprise a desired Shore hardness and, at the same time, a comparatively low density.

Preferred is a pad comprising a foamed pad main body, characterized in that the pad main body is formed from foamed styrene/butadiene-styrene (SBS) and/or foamed styrene-ethylene-butylene-styrene (SEBS) as the main component, comprises a hardness of at least 20 Shore OO and at most 45 Shore OO, and comprises a density of at least 0.4 g/cm³ and at most 0.8 g/cm³.

In a preferred embodiment, the pad main body is formed by introducing a propellant. The propellant may be a physical propellant or a chemical propellant.

Preferably, the propellant is a physical propellant.

The propellant can be a gas such as air, CO₂, nitrogen, or a mixture of gases.

Preferably, the propellant is CO₂ and/or nitrogen. Preferably, the propellant is CO₂ or nitrogen.

In a preferred embodiment, the propellant used for manufacturing is in the form of microspheres, i.e., it is a gas surrounded by a thermoplastic shell, for example, a polymer shell. Heating increases the gas pressure inside the shell, which softens at the same time. This increases the volume of the microspheres. However, the propellant gas remains permanently enclosed.

Preferably, the propellant thus comprises a gas, for example air, CO₂, nitrogen, or a mixture of gases, and a shell, for example a polymer shell. The pad main body may thus also comprise the components of the shell.

In an alternative embodiment, the propellant used for manufacturing is a granular or powdered propellant.

In a preferred embodiment, the propellant is incorporated in the manufacture of the pad main body in a quantity of at least 0.1 vol %, more preferred of at least 0.5 vol %. In a preferred embodiment, the propellant is incorporated in the manufacture of the pad main body in a quantity of at most 8 vol %, more preferred of at most 5 vol %.

The quantity of the propellant can be used advantageously to influence the density and thus the weight of the pad.

In a preferred embodiment, the pad main body comprises bubbles with a size of at least 30 μm. In a preferred embodiment, the pad main body comprises bubbles with a size of at most 300 μm. In a preferred embodiment, the pad main body comprises bubbles with a size of at least 35 μm. In a preferred embodiment, the pad main body comprises bubbles with a size of at most 275 μm. In a preferred embodiment, the pad main body comprises bubbles with a size of at least 40 μm. In a preferred embodiment, the pad main body comprises bubbles with a size of at most 250 μm.

In a preferred embodiment, the pad main body comprises bubbles with a size of 30 μm to 300 μm.

In a preferred embodiment, the pad main body comprises bubbles with a size of 35 μm to 275 μm.

In a preferred embodiment, the pad main body comprises bubbles with a size of 40 μm to 250 μm.

Preferably, the pad main body is a solidified foam. The foam structure can be closed-cell and/or open-cell. Surprisingly, it was found that the choice of propellant can influence the cell structure. With a physical propellant, the foam structure tends to be closed-cell. With a chemical propellant, the foam structure tends to be open-cell.

The pad main body may comprise additives from the manufacturing, such as polymers, oils, and waxes, particularly in smaller quantities.

The pad main body may comprise polymers and/or oils and/or waxes. The pad main body may comprise waxes in a weight quantity of up to 5 wt %, particularly up to 2 wt %.

The pad main body may comprise a dispersing oil, particularly in a weight quantity of 0.1 wt % to 4 wt %, preferably up to 2 wt %.

The pad main body may comprise a carrier material for the propellant, for example ethylene vinyl acetate (EVA), wax, and/or polybutylene. The pad main body may comprise the carrier material in a quantity of at most 10 wt %, preferably of at most 5 wt %, and particularly of at most 4 wt %.

The pad main body may comprise ethylene vinyl acetate (EVA), preferably in an amount of at most 10 wt %, preferably of at most 5 wt %, and particularly of at most 3.5 wt %.

According to the invention, the pad main body comprises a hardness of at least 20 Shore OO and at most 45 Shore OO. Preferably, the pad main body comprises a hardness of at least 23 Shore OO and at most 45 Shore OO. Preferably, the pad main body comprises a hardness of at least 23 Shore OO. Preferably, the pad main body comprises a hardness of at least 25 Shore OO and at most 45 Shore OO. Preferably, the pad main body comprises a hardness of at least 25 Shore OO. Preferably, the pad main body comprises a hardness of at least 26 Shore OO. Preferably, the pad main body comprises a hardness of at least 27 Shore OO. Preferably, the pad main body comprises a hardness of at least 28 Shore OO. Preferably, the pad main body comprises a hardness of at most 44 Shore OO. Preferably, the pad main body comprises a hardness of at most 43 Shore OO. Preferably, the pad main body comprises a hardness of at most 42 Shore OO. Preferably, the pad main body comprises a hardness of at most 34 Shore OO.

Preferably, the pad main body comprises a hardness of at least 27 Shore OO and at most 37 Shore OO. Preferably, the pad main body comprises a hardness of at least 28 Shore OO and at most 34 Shore OO.

According to the invention, the pad main body comprises a density of at least 0.4 g/cm³ and at most 0.8 g/cm³. According to the invention, the pad main body preferably comprises a density of at least 0.5 g/cm³ and at most 0.8 g/cm³. According to the invention, the pad main body preferably comprises a density of at least 0.4 g/cm³ and at most 0.7 g/cm³. According to the invention, the pad main body preferably comprises a density of at least 0.5 g/cm³ and at most 0.7 g/cm³.

It is advantageous to provide pads that comprise a suitable or desired viscoelasticity. Viscoelasticity refers to the time-, temperature-, and frequency-dependent elasticity of polymer melts or solids (plastics). Viscoelasticity is characterized by a partially elastic, partially viscous behavior. Viscoelastic materials comprise both a measurable storage modulus and a measurable loss modulus. If the storage modulus is greater than the loss modulus, the material is referred to as a solid; otherwise, it is referred to as a liquid. The pad main bodies can also be considered viscoelastic materials by specifying their storage and loss moduli, G′ and G′, or their loss factor tan delta=G″/G′.

Preferably, the pad main body comprises a storage modulus of at least 0.15 MPa in a temperature range of 18 °C to 40 °C. Preferably, the pad main body comprises a storage modulus of at least 0.20 MPa in a temperature range of 18 °C to 40 °C. Preferably, the pad main body comprises a storage modulus of 0 to at most 0.5 MPa in a temperature range of 18 °C to 40 °C. Preferably, the pad main body comprises a storage modulus of at most 0.4 MPa in a temperature range of 18 °C to 40 °C.

Preferably, the pad main body comprises a storage modulus of at least 0.15 MPa and at most 0.5 MPa in a temperature range of 18 °C to 40 °C. Preferably, the pad main body comprises a storage modulus of at least 0.15 MPa and at most 0.35 MPa in a temperature range of 18 °C to 40 °C.

Preferably, the pad main body comprises a loss modulus of at least 0.013 MPa in a temperature range of 10 °C to 40 °C. Preferably, the pad main body comprises a loss modulus of at least 0.015 MPa in a temperature range of 10 °C to 40 °C. Preferably, the pad main body comprises a loss modulus of at least 0.020 MPa in a temperature range of 10 °C to 40 °C. Preferably, the pad main body comprises a loss modulus of at most 0.03 MPa in a temperature range of 10 °C to 40 °C. Preferably, the pad main body comprises a loss modulus of at most 0.027 MPa in a temperature range of 10 °C to 40 °C. Preferably, the pad main body comprises a loss modulus of at least 0.015 MPa and at most 0.03 MPa in a temperature range of 10 °C to 40 °C. Preferably, the pad main body comprises a loss modulus of at least 0.015 MPa and at most 0.03 MPa in a temperature range of 10 °C to 40 °C. Preferably, the pad main body comprises a loss modulus of at least 0.013 MPa and at most 0.026 MPa in a temperature range of 10 °C to 40 °C.

Preferably, the pad main body comprises a loss factor tan delta of at least 0.05 in a temperature range from 10 °C to 40 °C. Preferably, the pad main body comprises a loss factor tan delta of at least 0.10 in a temperature range from 10 °C to 40 °C. Preferably, the pad main body comprises a loss factor tan delta of at most 0.25 in a temperature range of 10 °C to 40 °C. Preferably, the pad main body comprises a loss factor tan delta of at most 0.20 in a temperature range of 10 °C to 40 °C. Preferably, the pad main body comprises a loss factor tan delta of at least 0.05 and at most 0.25 in a temperature range of 10 °C to 40 °C. Preferably, the pad main body comprises a loss factor tan delta of at least 0.075 and at most 0.17 in a temperature range of 10 °C to 40 °C. Suitable rheological methods for measuring the storage modulus, loss modulus, and loss factor are known to the person skilled in the art. A preferred measurement is carried out with the test parameters 1 Hz, 25 °C, and 10 μm. DIN EN ISO 6721-12 can be used to select the measurement parameters. In a preferred embodiment, a covering and/or at least one active element are assigned to the pad main body. Suitable coverings, for example made of fabric or plastic, are known to the person skilled in the art. Coverings such as those used for conventional pads can be used here. Suitable active elements, for example pressure you/or massage elements, are known to the person skilled in the art. Active elements such as those used for conventional pads can be used here. Such elements are described, for example, in EP 0 496 071 A1 or DE 10 2019 212 740 B3. The pad according to the invention has the advantage that it can be used like a conventional pad or can replace it.

In the context of the present invention, a pad is understood to mean not only an orthopedic pad or a sports pad, but also a cushion or padding that is particularly assigned to an orthopedic aid or sports aid device and is used, for example, to exert pressure or for padding.

In a preferred embodiment, the pad is a knee pad, an ankle pad, a back pad, an elbow pad, a pad for the arm area, or a pad for the abdominal area. Suitable shapes and design options are known to the person skilled in the art.

In a preferred embodiment, the pad is not a shoe insole, foot insole, or shoe sole.

The present invention also relates to an orthopedic aid or sports aid device comprising a pad according to the invention.

Preferably, the orthopedic aid or sports aid device is an orthosis or bandage.

In a preferred embodiment, the orthosis or bandage is a knee orthosis or knee bandage, an ankle orthosis or bandage, a back orthosis or bandage, an elbow orthosis or bandage, an orthosis or bandage for the arm area, or an orthosis or bandage for the abdominal area.

Preferably, the orthosis is an elastic knitted orthosis or the bandage is an elastic knitted bandage.

Further preferred embodiments of the invention are set forth in the subclaims and the subsidiary claims.

The invention is described in more detail using the following example, without the embodiments of the invention described therein being understood as limiting.

### EXAMPLE

Manufacturing a pad from styrene block copolymers, for example styrene/butadiene-styrene (SBS) and/or styrene-ethylene-butylene-styrene (SEBS):

The following is used as the starting material:

Styrene/butadiene/styrene (SBS) and/or styrene-ethylene-butylene-styrene (SEBS) is used as the base material in the form of a gel mixture with white oil in a weight quantity of 94% to 99.8%, for example a gel from Hexpol® such as Dryflex Gel, i.e. an SEBS-oil mixture consisting of approx. 10% SEBS and 90% medical white oil, as well as additives.

Oil, for example dispersing oil in a weight quantity of 0.1% to 2%.

Propellant microspheres (5 μm-40 μm) in a weight quantity of 0.1% to 4%, for example Expancel® DU (951 DU 120) from Hoesch®.

In a method known to the person skilled in the art, microspheres of a certain size are drummed onto the base material to be foamed using a dispersing oil. Here, the surface of the granulate grains of the base material is first wetted with a so-called dispersing oil and then the microspheres are powdered in powder form at 0.1-4%. The microspheres adhere to the base material due to the granulate grain surface being wetted with oil. This results in a covered granulate.

During dosing on an injection molding machine, the base material is drawn into the cylinder via the screw with the drummed-on propellant, where it is melted and mixed. During dosing and melting (160 °C-230 °C), the activation temperature (80 °C-140 °C) of the microspheres is exceeded, causing them to expand. The base material with the drummed-on propellant is converted into a melt-foam phase in the injector. The screw is then retracted slightly to allow the molten foam to expand (expansion factor of the foam: 5%-100%). The molten foam is then injection molded into the mold cavity to create the desired shape of the pad base. The molten foam cools down to 40 °C-20 °C) in the mold and is then removed from the mold cavity. The result is a pad main body in the desired shape with a hardness of approximately 30 to 35 Shore OO and a density between 0.4 g/cm³ and 0.8 g/cm³.

1. An orthopedic pad comprising a foamed pad main body, wherein the foamed pad main body is formed from a thermoplastic elastomer (TPE) as the main component, the thermoplastic (TPE) comprises a hardness of at least 20 Shore OO and at most 45 Shore OO, comprises a density of at least 0.4 g/cm³ and at most 0.8 g/cm³, comprises a storage modulus of at least 0.15 MPa and at most 0.5 MPa in a temperature range of at least 18 °C and at most 40 °C, comprises a loss modulus of at least 0.015 MPa and at most 0.03 MPa in a temperature range of at least 10 °C and at most 40 °C, and comprises a loss factor tan delta of at least 0.05 and at most 0.25 in a temperature range of at least 10 °C and at most 40 °C; and
wherein the foamed pad main body is formed by introducing a propellant and wherein the foamed pad main body comprises bubbles with a size of 30 μm to 300 μm.

2. The orthopedic pad of claim 1, wherein the thermoplastic elastomer is selected from the group consisting of styrene block copolymers (TPS), thermoplastic polyurethanes (TPU), thermoplastic vulcanizates (TPV), thermoplastic polyamide elastomers (TPA), thermoplastic copolyester elastomers (TPC), and mixtures thereof.

3. The orthopedic pad of claim 1, wherein the thermoplastic elastomer (TPE) comprises is at least one styrene block copolymer (TPS).

4. The orthopedic pad of claim 3, wherein the at least one styrene block copolymer (TPS) is formed on the basis of styrene/butadiene-styrene (SBS) and/or styrene-ethylene-butylene-styrene (SEBS).

5. The orthopedic pad of claim 1, wherein the foamed pad main body comprises bubbles with a size of 40 μm to 250 μm.

6. The orthopedic pad of claim 1, wherein the pad main body is assigned a covering and/or at least one active element.

7. The orthopedic pad of claim 1, wherein the orthopedic pad is not a shoe insole or shoe sole.

8. An orthopedic aid or sports aid device comprising the orthopedic pad of claim 1.

9. The orthopedic aid or sports aid device of claim 8, wherein the orthopedic aid or sports aid device is an orthosis or bandage. (New) The orthopedic pad of claim 1, wherein the foam structure of the foamed pad main body is a closed-cell structure.

11. The orthopedic pad of claim 1, wherein the foam structure of the foamed pad main body is an open-cell structure.

12. The orthopedic pad of claim 6, wherein the propellant is a physical propellant selected from CO₂ and nitrogen.

13. The orthopedic pad of claim 6, wherein the propellant comprises expandable microspheres comprising a gas enclosed by a thermoplastic shell.

14. The orthopedic pad of claim 13, wherein the expandable microspheres are present during manufacture in a quantity of 0.5 vol % to 5 vol %.

15. The orthopedic pad of claim 1, wherein the foamed pad main body comprises an oil or an oil mixture in an amount of 50 wt % to 95 wt %, wherein the oil or oil mixture comprises a white oil.

16. The orthopedic pad of claim 6, wherein a carrier material for the propellant selected from ethylene vinyl acetate (EVA), wax, and polybutylene is present in the foamed pad main body in an amount of at most 5 wt %.

17. A method of manufacturing an orthopedic pad, comprising:

18. method of claim 17, wherein the propellant comprises microspheres selected such that, upon heating, the gas pressure within the thermoplastic shell increases and the shell softens to allow volume expansion, and wherein the dispersing oil is present in an amount of 0.1 wt % to 2 wt % and the microspheres are present in an amount of 0.1 wt % to 4 wt % during manufacture.

19. The orthopedic aid or sports aid device of claim 8, wherein the device is selected from a knee orthosis or knee bandage, an ankle orthosis or ankle bandage, a back orthosis or back bandage, an elbow orthosis or elbow bandage, an orthosis or bandage for the arm area, and an orthosis or bandage for the abdominal area.

20. The orthopedic aid or sports aid device of claim 8, wherein the device comprises an elastic knitted orthosis or an elastic knitted bandage and the orthopedic pad is embedded in a covering and/or assigned
